(19) United States
(12) Patent Application Publication
(10) Pub. No.: US 2022/0361799 A1
(43) Pub. Date: Nov. 17, 2022

(54) CIRCUITLESS HEART CYCLE DETERMINATION

(71) Applicant: Caption Health, Inc., Brisbane, CA (US)

(72) Inventors: Ha Hong, Pleasant Hill, CA (US); Kilian Koepsell, San Francisco, CA (US); Nicolas Poilvert, Seattle, WA (US)

(21) Appl. No.: 17/321,269

(22) Filed: May 14, 2021

(51) Int. Cl. A61B 5/352; A61B 5/00; A61B 5/335

(52) U.S. Cl. CPC A61B 5/352; A61B 5/7267; A61B 5/335

(57) ABSTRACT

Circuitless heart cycle determination includes capturing a video clip of one or more image frames of a target heart muscle through an ultrasound imaging device and submitting the frames to a classifier that has been trained with an annotated set of images, each of a corresponding heart muscle captured at a specified phase of a heart cycle with a ground truth indication of the specified phase of the heart cycle drawn from a separately recorded cycle graph of an electrical signal measured over time for the corresponding heart muscle. In response to the submission, a classification is received of different portions of the submitted frames according to corresponding phases of the heart cycle. Finally, a contemporaneous phase of the heart cycle is determined in the device for the target heart muscle without sensing electrical signals by way of a closed-loop sensor circuit affixed proximately to the target heart muscle.

### BACKGROUND OF THE INVENTION

### Field of the Invention

The present invention relates to the field of cardiac monitoring and more particularly to the computational characterization of a heart cycle.

### Description of the Related Art

Cardiac monitoring generally refers to continuous or intermittent monitoring of heart activity, generally by electrocardiography, with assessment of the patient's condition relative to their cardiac rhythm. Generally, cardiac monitoring requires the affixation of two or more different sensors upon the surface of the body so as to create individual two-sensor electrical circuits. Then, as the electrical signals imparted upon different portions of a monitored heart muscle intensify and then wane, different pairs of the sensors forming different circuits measure different aspects of what is known as the cardiac cycle or heart cycle. The foregoing is known as an electrocardiogram, abbreviated as “ECG” or “EKG”.

The cardiac cycle, or heart cycle, is the performance of the human heart from the beginning of one heartbeat to the beginning of the next heartbeat. The cycle consists of two periods: one during which the heart muscle relaxes and refills with blood, referred to as diastole, following a period of contraction and the pumping of blood, referred to as systole. The heart cycle is known to include five different phases of which three are generally visible in a graph representative of the heart cycle, and in most cases all five are visible. The first phase of the heart cycle is the P-cycle representative of the conduction of electrical impulses to the atria of the heart causing the atria to contract followed by a brief delay and then the QRS complex portion of the heart cycle, which reflects the spread of electrical activity through the ventricular myocardium and inherently includes three of the five phases of the heart cycle and is known as the R-wave portion. Thereafter, following another short delay, the T-cycle commences and reflects the repolarization of the ventricles thereby restoring the heart into a resting state.

The graph representative of the heart cycle generally is the product of the ECG. However, there are many occasions when processing the heart cycle can be important outside of the conduct of an ECG for instance knowing a contemporaneous moment during the heart cycle can be advantages when performing a cardiac ultrasound. However, the quality of an ECG depends upon the proper placement of electrodes upon the patient and the resulting quality of contact between electrode and patient. As well, during a cardiac ultrasound, where it is necessary to manipulate an ultrasound wand, the necessity of different sensors placed upon the chest of the patient from which a multiplicity of wires can be unwieldy and modern ultrasound units lack an internal ECG capability. Yet, knowing the heart cycle evident from an ECG can be helpful in improving the quality of imagery produced during ultrasound.

### BRIEF SUMMARY OF THE INVENTION

Embodiments of the present invention address deficiencies of the art in respect to heart cycle determination during cardiac ultrasound and provide a novel and non-obvious method, system and computer program product for circuitless heart cycle determination during cardiac ultrasound. A method for a circuitless heart cycle determination includes capturing a video clip of one or more image frames of a target heart muscle through a cardiac ultrasound imaging device, and submitting the one or more image frames to a classifier that has been trained with an annotated set of images, each of a corresponding heart muscle captured at a specified phase of a heart cycle with a ground truth indication of the specified phase of the heart cycle drawn from a separately recorded cycle graph of an electrical signal measured over time for the corresponding heart muscle.

The method additionally includes receiving in response to the submission, a classification of different portions of the submitted image frames according to corresponding phases of the heart cycle. Finally, the method includes presenting an indication in the cardiac ultrasound imaging device, of a contemporaneous one of the corresponding phases of the heart cycle for the target heart muscle. Importantly, the contemporaneous phase of the heart cycle for the target heart muscle is determined in the ultrasound imaging device without sensing electrical signals by way of a closed-loop sensor circuit affixed proximately to the target heart muscle. In this way, the determined heart cycle can be used in order to further acquisition of quality ultrasound imagery by the cardiac ultrasound imaging device.

To that end, in one aspect of the embodiment, the classification of the captured imagery is the classification of a portion of the frames of the video clip corresponding to an R-wave portion of the cycle graph measured over the heart cycle. In another aspect of the embodiment, the classification of the captured imagery is the classification of a portion of the frames of the video clip corresponding to an R-to-R interval of the cycle graph measured over multiple different heart cycles. In respect to the latter, a number of the different heart cycles may be specified so that the frames of the video clip may be clipped to include only portions of the frames of the video clip corresponding to the R-to-R interval for the specified number of the different heart cycles. Then, playback of the clipped frames may be looped in order to provide a periodic view of the clipped frames in support of a specific medical diagnosis while excluding other portions of the frames not pertinent to the diagnosis.

In another aspect of the embodiment, the method additionally includes identifying portions of the frames of the video clip corresponding to an R-wave while removing remaining other portions of the frames of the video clip. In even yet another aspect of the embodiment, a model curve may be generated from the separately recorded cycle graph. Thereafter, the generated contemporaneous cycle graph may be curve fitted to the model curve in order to smooth the generated graph and to more accurately present the generated graph.

In another embodiment of the invention, a data processing system is adapted for circuitless heart cycle determination during cardiac ultrasound. The system includes a host computing platform of one or more computers, each with memory and at least one processor. The system also includes a heart cycle determination module. The module in turn includes computer program instructions enabled while executing in the host computing platform to submit a captured video clip of one or more image frames of a target heart muscle to a classifier trained with an annotated set of images each of a corresponding heart muscle captured at a specified phase of a heart cycle with a ground truth indication of the specified phase of the heart cycle drawn from a separately recorded cycle graph of an electrical signal measured over time for the corresponding heart muscle. The instructions further are enabled to receive in response to the submission, a classification of different portions of the submitted frames according to corresponding phases of the heart cycle. Finally, the instructions are enabled to present an indication in the cardiac ultrasound imaging device, of a contemporaneous cycle graph for the target heart muscle without sensing electrical signals by way of a closed-loop sensor circuit affixed proximately to the target heart muscle.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for circuitless heart cycle determination during cardiac ultrasound;

FIG. 2 is a schematic diagram of a data processing system adapted for circuitless heart cycle determination during cardiac ultrasound; and,

FIG. 3 is a flow chart illustrating a process for circuitless heart cycle determination during cardiac ultrasound.

### DETAILED DESCRIPTION OF THE INVENTION

Embodiments of the invention provide for circuitless heart cycle determination during cardiac ultrasound. In accordance with an embodiment of the invention, different ECGs are acquired contemporaneously with ultrasound imaging of correspondingly different hearts over multiple different heart cycles. Different video clips each of one or more image frames of the ultrasound imaging are then annotated with different phases of the heart cycle occurring contemporaneously with the different image frames as determined from a corresponding one of the ECGs. The annotated frame or frames are then provided to a classifier so as to associate pixel elements of the annotated frame or frames with a correspondingly annotated one of the different phases of the heart cycle. Thereafter, in consequence of the conduct of an ultrasound examination of a target heart, the acquired frame or frames of the target heart can be submitted to the classifier in order to receive in response, a prediction of a contemporaneous phase of the heart cycle of the target heart based upon the pixel elements of the acquired frame or frames and without the use of one or more sensor pairs of an ECG.

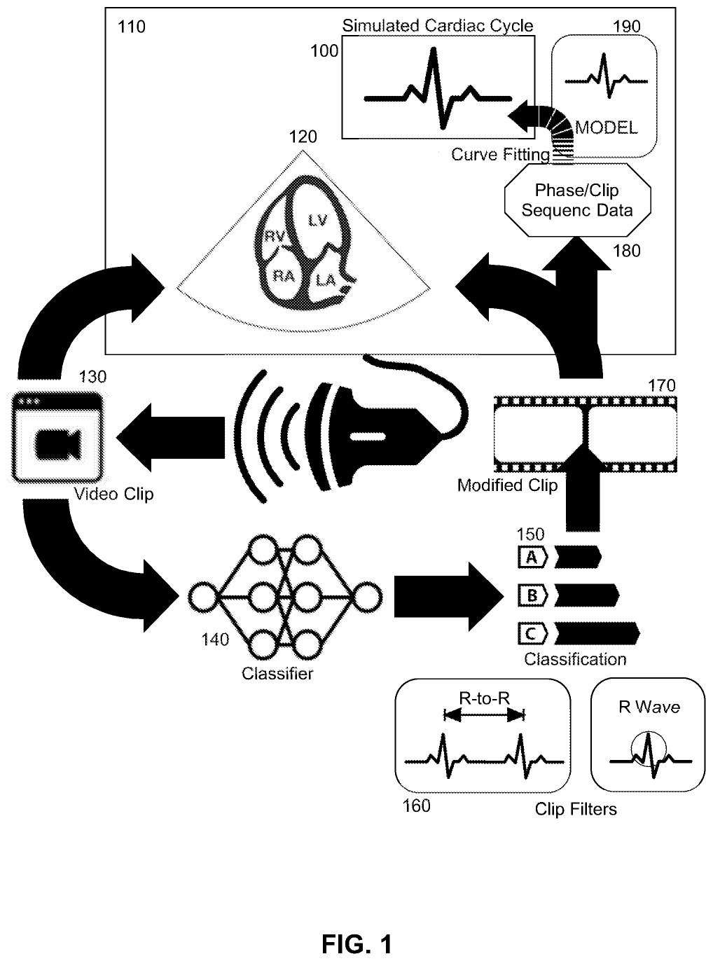

In further illustration, FIG. 1 pictorially shows a process for circuitless heart cycle determination. As shown in FIG. 1, an ultrasound diagnostic imaging device 110 acquires video clip imagery 130 of one or more image frames of a target heart. Thereafter, the device 110 submits the frame or frames of the video clip imagery 130 to a classifier 140. The classifier 140, in turn, processes the individual pixels of the different frames of the video clip imagery 130, and produces a classification 150 of the frame or frames of the video clip imagery 130 as belonging to one of several phases of a heart cycle. The classification 150 may then be presented within a display of the device 110. As well, the classifier 140 produces a set of data points 180 reflective of both a phase determined for a corresponding portion of the frame or frames of the video clip imagery 130 and time sequence values for different ones of the frames in a sequence of the frames of the video clip imagery 130

Once the classification 150 has been determined for the frame or frames of the video clip imagery 130, a modified set of frame or frames 170 can be produced by excluding from the modified frames 170, different ones of the image frames of the video clip imagery 130 with a classification 150 filing outside of a filter 160, such as a filter excluding classifications outside of an R-wave phase of the cardiac cycle, or a classification outside a specific R-to-R portion of the cardiac cycle. The device 110 then presents a display 120 of the modified frames 170 in the device 110. As well, as an option, the data points 180 from the video clip imagery 130 are fit against an ECG model 190 to produce a simulated ECG display 100 for presentation in the device 110.

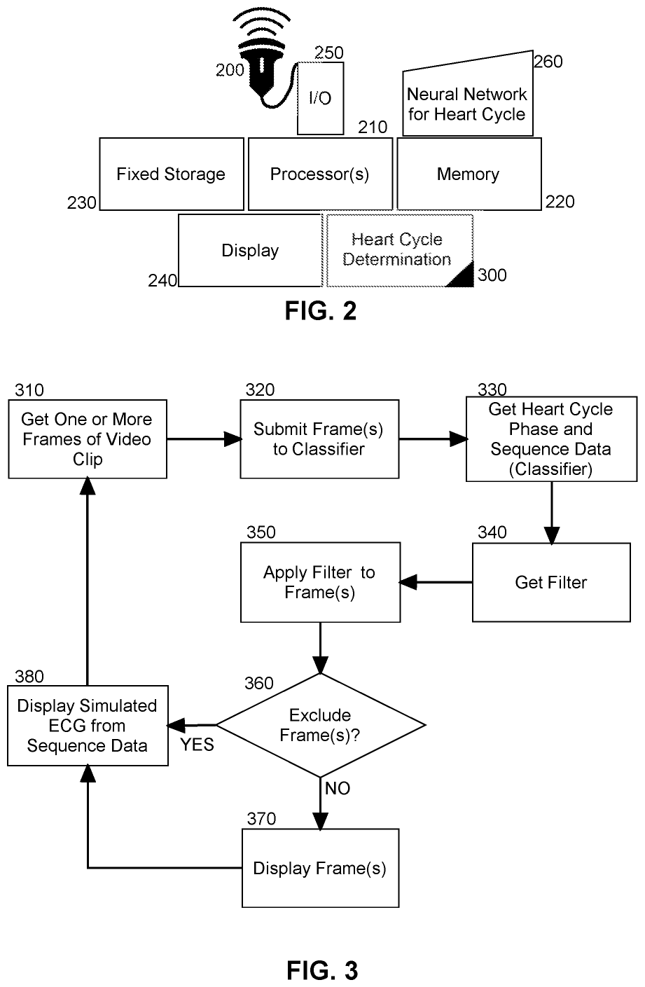

The process described in connection with FIG. 1 may be implemented within a computer data processing system. In further illustration, FIG. 2 schematically shows a data processing system adapted for circuitless heart cycle determination. The system includes a host computing platform that includes one or more computers, each with at least one processor 210, memory 220, fixed storage 230 and a display 240. The fixed storage 230 stores therein one or more frames of respective ultrasound video clips of a target heart acquired by input/output circuitry 250 communicatively coupled to an ultrasound diagnostic imaging device 200.

A neural network 260 may be loaded at run time into the memory 220 of the host computing platform. The neural network 260 is trained to correlate different imagery of different image frames of different video clips of different hearts with different cardiac cycle phases so that when the neural network 260 is presented with a contemporaneously acquired set of frames of a video clip of the target heart, the neural network 260 returns a correlated cardiac cycle phase such as P-wave, R-wave or T-wave. Likewise, the neural network 260 is trained to correlate different image frames of different video clips of different hearts with corresponding classification data points so that when the neural network 260 is presented with the contemporaneously acquired frame or frames of a video clip of the target heart, the neural network 260 also returns a correlated sequence of classification data points which may then be curve fit into a simulated ECG.

Of note, the system yet further includes a heart cycle determination module 300. The heart cycle determination module 300 includes computer program instructions that when executing in the memory 220 by the one or more processors 210 of the host computing platform, loads into the memory 220 the neural network 260 and receives a continuous stream of different frames of respectively different video clips, either previously acquired and stored in the fixed storage 230, or contemporaneously acquired in real time from the ultrasound diagnostic imaging device 200, and submits the frames of the video clips in succession to the neural network 260. Thereafter, the program instructions receive from the neural network 260 in response, a classification of each of the frames of the video clips as to a corresponding phase of the heart cycle and sequencing data for the classification. The program instructions then curve fit the sequenced data against a known model of an ECG in order to produce a simulated ECG that can be displayed in the display 240.

As well, the program instructions extract from the frame or frames of the video clip, portions of the frames of the video clips having been classified outside of a specified filter. By way of example, a filter can exclude any frame or frames of the video clip imagery classified outside of an R-wave portion of the heart cycle or video clip imagery classified as falling outside of a threshold number of R-to-R heart cycles. Optionally, the program code extracts from the frame or frames of the video clip, all frames associated with portions of the heart cycle outside of a single complete R-to-R cycle and then displays a looping presentation of the remaining frame or frames of the video imagery in the display 240.

In even yet further illustration of the operation of the heart cycle determination module 300, FIG. 3 is a flow chart illustrating a process for circuitless heart cycle determination. Beginning in block 310, a video clip of one or more image frames is received from an ultrasound diagnostic imaging device and in block 320, the received frame or frames is submitted to a classifier. In block 330, both a heart cycle phase, and also sequencing data of different frames and corresponding classifications are received from the classifier. Then, in block 340, a filter is retrieved and in block 350, the filter is applied to the frames in order to exclude from display those of the frames classified as being outside of the filter based upon an association with a particular portion of the heart cycle phase. As such, in decision block 360, it is determined whether or not to exclude the frame or frames from display in the ultrasound diagnostic imaging device.

If it is determined not to exclude the frame or frames from display in the ultrasound diagnostic imaging device, the frame or frames is displayed in the ultrasound diagnostic imaging device in block 370. In either circumstance, however, in block 380 the sequencing data is fitted with a model ECG graph in order to produce a simulated ECG which then is displayed in the ultrasound diagnostic imaging device. Thereafter, the process repeats for a next received video clip. In this way, the heart cycle determination association with each frame of the video clip can be used in order to determine which of the frames of the video clip to exclude from view in so far as higher quality image frames are correlated to certain phases of the heart cycle.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “include”, “includes”, and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for a circuitless heart cycle determination during cardiac ultrasound comprising:
submitting a cardiac ultrasound video clip of one or more image frames of a target heart muscle to a classifier trained with an annotated set of images each of a corresponding heart muscle captured at a specified phase of a heart cycle with a ground truth indication of the specified phase of the heart cycle drawn from a separately recorded cycle graph of an electrical signal measured over time for the corresponding heart muscle;
receiving in response to the submission, a classification of different portions of the submitted one or more image frames according to corresponding phases of the heart cycle; and,
determining a contemporaneous one of the corresponding phases of the heart cycle for the target heart muscle without sensing electrical signals by way of a closed-loop sensor circuit affixed proximately to the target heart muscle.

2. The method of claim 1, wherein the classification of the captured imagery is the classification of a portion of the one or more image frames corresponding to an R-wave portion of the cycle graph measured over the heart cycle.

3. The method of claim 1, wherein the classification of the captured imagery is the classification of a portion of the one or more image frames corresponding to an R-to-R interval of the cycle graph measured over multiple different heart cycles.

4. The method of claim 1, further comprising identifying portions of the one or more image frames corresponding to an R-wave and removing remaining other portions of the one or more image frames.

5. The method of claim 3, further comprising specifying a number of the different heart cycles, clipping the one or more image frames to include only portions of the one or more image frames corresponding to the R-to-R interval for the specified number of the different heart cycles and looping playback of the clipped one or more image frames.

6. The method of claim 1, further comprising generating a model curve from the separately recorded cycle graph and curve fitting a multiplicity of contemporaneous ones of the phases of the heart cycle for the target heart muscle to the model curve so as to produce a simulated graph.

7. A data processing system adapted for circuitless heart cycle determination during cardiac ultrasound, the system comprising:
a host computing platform comprising one or more computers, each comprising memory and at least one processor; and,
a heart cycle determination module comprising computer program instructions enabled while executing in the host computing platform to perform:
capturing through a cardiac ultrasound imaging device, a video clip of one or more image frames of a target heart muscle;
submitting the one or more image frames to a classifier trained with an annotated set of images each of a corresponding heart muscle captured at a specified phase of a heart cycle with a ground truth indication of the specified phase of the heart cycle drawn from a separately recorded cycle graph of an electrical signal measured over time for the corresponding heart muscle;
receiving in response to the submission, a classification of different portions of the submitted one or more image frames according to corresponding phases of the heart cycle; and,
determining a contemporaneous one of the corresponding phases of the heart cycle for the target heart muscle without sensing electrical signals by way of a closed-loop sensor circuit affixed proximately to the target heart muscle.

8. The system of claim 7, wherein the classification of the captured imagery is the classification of a portion of the one or more image frames corresponding to an R-wave portion of the cycle graph measured over the heart cycle.

9. The system of claim 7, wherein the classification of the captured imagery is the classification of a portion of the one or more image frames corresponding to an R-to-R interval of the cycle graph measured over multiple different heart cycles.

10. The system of claim 7, further comprising identifying portions of the one or more image frames corresponding to an R-wave and removing remaining other portions of the one or more image frames.

11. The system of claim 9, further comprising specifying a number of the different heart cycles, clipping the one or more image frames to include only portions of the one or more image frames corresponding to the R-to-R interval for the specified number of the different heart cycles and looping playback of the clipped one or more image frames.

12. The system of claim 7, wherein the program instructions further perform generating a model curve from the separately recorded cycle graph and curve fitting a multiplicity of contemporaneous ones of the corresponding phases of the heart cycle for the target heart muscle to the model curve so as to produce a simulated graph.

13. A computer program product for circuitless heart cycle determination during cardiac ultrasound, the computer program product including a computer readable storage medium having program instructions embodied therewith, the program instructions executable by a device to cause the device to perform a method including:
capturing through a cardiac ultrasound imaging device, a video clip of one or more image frames of a target heart muscle;
submitting the one or more image frames to a classifier trained with an annotated set of images each of a corresponding heart muscle captured at a specified phase of a heart cycle with a ground truth indication of the specified phase of the heart cycle drawn from a separately recorded cycle graph of an electrical signal measured over time for the corresponding heart muscle;
receiving in response to the submission, a classification of different portions of the submitted one or more image frames according to corresponding phases of the heart cycle; and,
determining a contemporaneous one of the corresponding phases of the heart cycle for the target heart muscle without sensing electrical signals by way of a closed-loop sensor circuit affixed proximately to the target heart muscle.

14. The computer program product of claim 13, wherein the classification of the captured imagery is the classification of a portion of the one or more image frames corresponding to an R-wave portion of the cycle graph measured over the heart cycle.

15. The computer program product of claim 13, wherein the classification of the captured imagery is the classification of a portion of the one or more image frames corresponding to an R-to-R interval of the cycle graph measured over multiple different heart cycles.

16. The computer program product of claim 13, wherein the method further comprises identifying portions of the one or more image frames corresponding to an R-wave and removing remaining other portions of the one or more image frames.

17. The computer program product of claim 15, wherein the method further comprises specifying a number of the different heart cycles, clipping the one or more image frames to include only portions of the one or more image frames corresponding to the R-to-R interval for the specified number of the different heart cycles and looping playback of the clipped one or more image frames.

18. The computer program product of claim 13, wherein the method further comprises generating a model curve from the separately recorded cycle graph and curve fitting a multiplicity of contemporaneous ones of the corresponding phases of the heart cycle for the target heart muscle to the model curve so as to produce a simulated graph.
